(19) United States
(12) Patent Application Publication
(10) Pub. No.: US 2021/0349069 A1
(43) Pub. Date: Nov. 11, 2021

(54) MONITORING DEVICE, PLANT GROWTH MONITORING METHOD USING MONITORING DEVICE, AND PLANT FACTORY

(71) Applicant: HONGFUJIN PRECISION ELECTRONICS(TIANJIN)CO.,LTD., Tianjin (CN)

(72) Inventors: CHIA-EN LI, New Taipei (TW); PO-HUI LU, Chu-Nan (TW); CHIEN-HAO SU, Chu-Nan (TW)

(21) Appl. No.: 17/017,458

(22) Filed: Sep. 10, 2020

(30) Foreign Application Priority Data
May 8, 2020 (CN) 202010381964.0

(51) Int. Cl. G01N 33/00; A01G 9/26; G06Q 50/02

(52) U.S. Cl. CPC G01N 33/0098; A01G 9/249; G06Q 50/02; A01G 9/26

(57) ABSTRACT

A plant growth monitoring method includes sensing growth data of a plant, determining reference data of the growth data, determining a first effective range of an N number of parameters of the reference data, determining a second effecting range of each parameter, and obtaining standard values of the N number of parameters in each of T growth periods.

### FIELD

The subject matter herein generally relates to monitoring devices, and more particularly to a monitoring device for monitoring the growth of a plant and a plant growth monitoring method using the monitoring device.

### BACKGROUND

Plant factories have stable mass production methods to produce crops. However, in plant factories, how to cultivate plants intelligently during their growth is a technical problem to be solved.

### BRIEF DESCRIPTION OF THE DRAWINGS

Implementations of the present disclosure will now be described, by way of embodiments, with reference to the attached figures.

FIG. 1A is a schematic block diagram of an embodiment of a monitoring device.

FIG. 1B is a schematic diagram of an embodiment of a plant factory.

FIG. 2 is a schematic block diagram of an embodiment of a monitoring system.

FIG. 3 is a flow chart diagram of a plant growth monitoring method.

### DETAILED DESCRIPTION

It will be appreciated that for simplicity and clarity of illustration, where appropriate, reference numerals have been repeated among the different figures to indicate corresponding or analogous parameters. Additionally, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. The drawings are not necessarily to scale and the proportions of certain parts may be exaggerated to better illustrate details and features. The description is not to be considered as limiting the scope of the embodiments described herein.

Several definitions that apply throughout this disclosure will now be presented.

The term “comprising” means “including, but not necessarily limited to”; it specifically indicates open-ended inclusion or membership in a so-described combination, group, series, and the like.

In general, the word “module” as used hereinafter refers to logic embodied in hardware or firmware, or to a collection of software instructions, written in a programming language such as, for example, Java, C, or assembly. One or more software instructions in the modules may be embedded in firmware such as in an erasable-programmable read-only memory (EPROM). It will be appreciated that the modules may comprise connected logic units, such as gates and flip-flops, and may comprise programmable units, such as programmable gate arrays or processors. The modules described herein may be implemented as either software and/or hardware modules and may be stored in any type of computer-readable medium or other computer storage device.

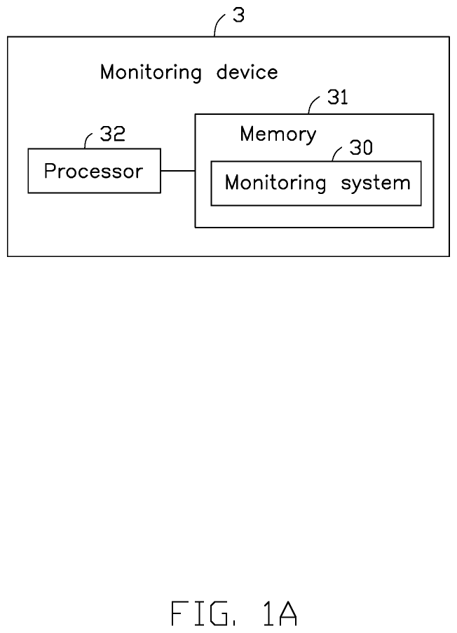

FIG. 1A shows a structural diagram of an embodiment of a monitoring device.

In one embodiment, the monitoring device 3 includes, but is not limited to, a memory 31 and at least one processor 32 electrically connected to the memory 31.

Those skilled in the art should understand that the structure of the monitoring device 3 shown in FIG. 1A does not constitute a limitation of the embodiment of the present disclosure. The monitoring device 3 may also include more or less hardware or software than those shown in FIG. 1A, or have different component arrangements.

It should be noted that the monitoring device 3 is only an example. If other existing or future monitoring devices can be adapted to the present disclosure, they should also be included in the protection scope of the present disclosure and included herein by reference.

In some embodiments, the memory 31 may be used to store program codes and various data of computer programs. For example, the memory 31 may be used to store a monitoring system 30 installed in the monitoring device 3 and realize high-speed and automatic access to programs or data during the operation of the monitoring device 3. The memory 31 may include a read-only memory, a programmable read-only memory, an erasable programmable read-only memory, a one-time programmable read-only memory, an electronically erasable programmable read-only memory, a compact disc read-only memory, or other optical disk storage, magnetic disk storage, tape storage, or any other non-volatile computer-readable storage medium that can be used to carry or store data.

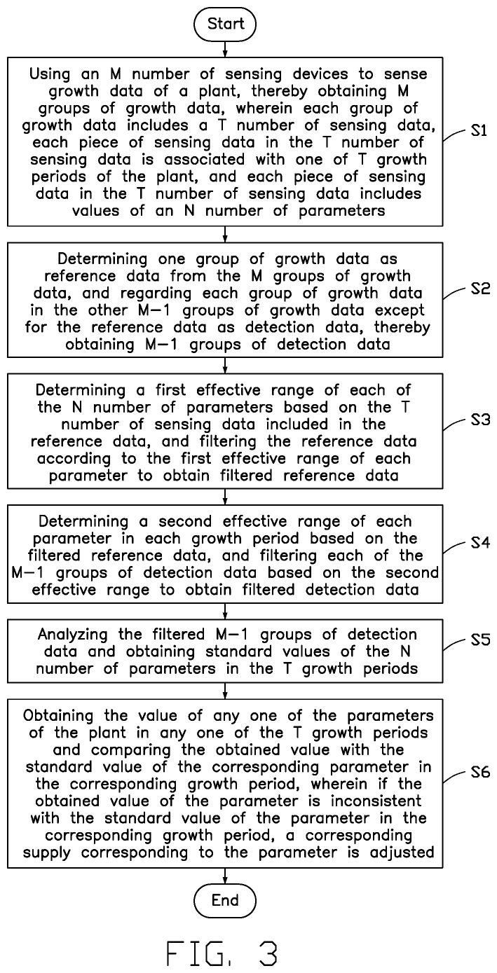

In some embodiments, the at least one processor 32 may include an integrated circuit. For example, the at least one processor 32 can include a single packaged integrated circuit, or a plurality of integrated circuits with the same function or different functions, including one or more central processing units, microprocessors, combinations of digital processing chips, graphics processors, and various control chips. The at least one processor 32 is a control core of the monitoring device 3, which uses various interfaces and lines to connect the various components of the entire monitoring device 3, and executes programs, instructions, or modules stored in the memory 31, and calls the data stored in the memory 31 to perform various functions of processing data of the monitoring device 3, for example, the function of analyzing and monitoring plant growth (refer to FIG. 3 for details).

Referring to FIG. 1B, in one embodiment, a plant factory 100 includes an M number of sensing devices 33. The M number of sensing devices 33 may each communicate with the monitoring device 3 in a wired or wireless communication manner. In other words, the M number of sensing devices 33 can be built in the monitoring device 3 or externally connected to the monitoring device 3. In one embodiment, M is a positive integer greater than or equal to 2. The value of M can be determined according to the number or area of plants 4 planted by the plant factory 100. For example, when one sensing device 33 is provided for one plant 4, the value of M is determined according to the number of plants 4.

In another example, when one sensing device 33 is provided for multiple plants 4 planted in the same area, the value of M is determined according to the number of areas where plants 4 are planted.

In one embodiment, each sensing device 33 is used to sense values of an N number of parameters of the plant 4 in each growth period. The N number of parameters include, but are not limited to, temperature, humidity, and brightness of the environment where the plant 4 is located, as well as nutrient components of the soil of the plant 4 such as nitrogen, phosphorus, and potassium. FIG. 1B illustrates two sensing devices 33, and each sensing device 33 senses the values of various parameters of one plant 4 in each growth period correspondingly.

In one embodiment, T growth periods can be defined according to the growth cycle of the plant 4 (T is a positive integer). For example, taking the growth cycle of the plant 4 including germination, growth, flowering, and fruiting as an example, the growth cycle of the plant 4 can be divided into four growth periods (that is, T is equal to 4). A first growth period is the germination period, a second growth period is the growth period, a third growth period is the flowering period, and a fourth growth period is the fruiting period. In other embodiments, the growth cycle can be divided into more or fewer growth periods.

In one embodiment, each sensing device 33 may include a temperature sensor, a humidity sensor, a light sensor, a soil nutrient sensor, and the like. The temperature sensor is used to sense the temperature of the environment where the plant 4 is located. The humidity sensor is used to sense the humidity of the environment where the plant 4 is located. The light sensor is used to sense the brightness of the environment where the plant 4 is located. The soil nutrient sensor is used to sense the nutrient content of the soil of the plant 4, such as nitrogen, phosphorus, potassium, and the like. Thus, each sensing device 33 can be used to sense the values of the N number of parameters of the plant 4 in each growth period.

In one embodiment, N is equal to six, and the six parameters include temperature, humidity, brightness, nitrogen content, phosphorus content, and potassium content. It should be noted that in other embodiments, there may be fewer or more parameters.

Referring to FIG. 1B, in one embodiment, the plant factory 100 may further include one or more supply devices 41 for adjusting the N number of parameters to the plants 4. The supply device 41 includes, but is not limited to, a heating device, a humidification device, a lighting device, and a nutrient supply device for regulating nutrients such as nitrogen, phosphorus, and potassium for the soil. The supply device 41 may communicate with the at least one processor 32 in a wired or wireless communication manner.

In one embodiment, the monitoring system 30 may include one or more modules, and the one or more modules are stored in the memory 31 and executed by the processor 32 to analyze and monitor plant growth (refer to FIG. 3 for details).

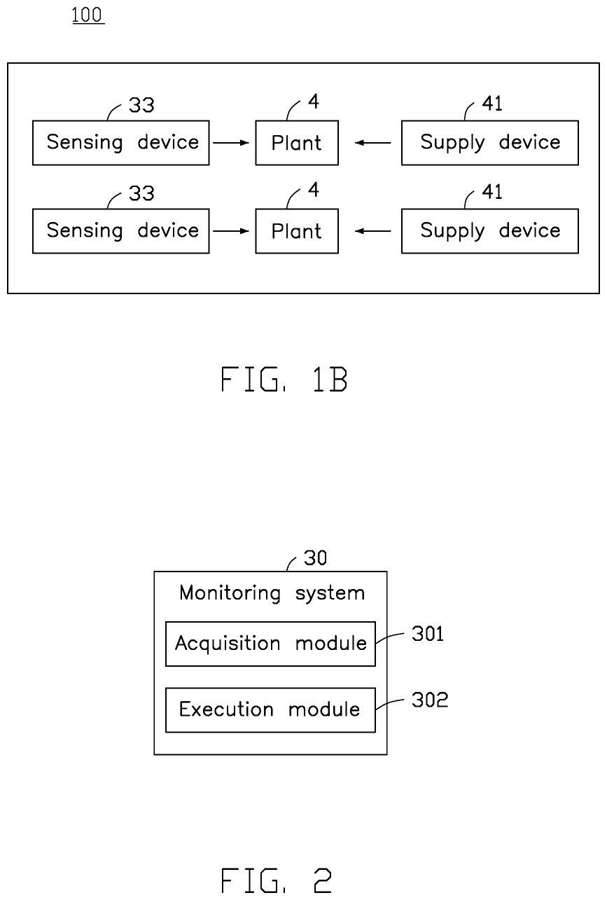

Referring to FIG. 2, the one or more modules include an acquisition module 301 and an execution module 302.

In one embodiment, the memory 31 stores program codes of a computer program, and the processor 32 executes the program codes stored in the memory 31 to perform related functions. For example, the various modules of the monitoring system 30 in FIG. 2 are program codes stored in the memory 31 and executed by the processor 32 so as to realize the functions of the various modules to achieve monitoring and control of plant growth.

FIG. 3 is a flowchart of a plant growth monitoring method provided by an embodiment of the present disclosure.

In one embodiment, the plant growth monitoring method can be applied to the monitoring device 3 for monitoring plant growth. The method may be implemented on the monitoring device 3 or in the form of a software development kit (SDK).

According to different requirements, a sequence of blocks of the plant growth monitoring method can be changed, and some blocks can be omitted or combined.

At block S1, the acquisition module 301 uses the M number of sensing devices 33 to sense the growth data of the plant 4, and obtains M groups of growth data. Each group of the growth data includes T number of sensing data. Each piece of sensing data in the T number of sensing data is associated with one of the T growth periods of the plant 4, and each piece of sensing data in the T number of sensing data includes the values of the N number of parameters.

As mentioned above, each sensing device 33 is used to sense the values of the N number of parameters of the plant 4 in each growth period.

In one embodiment, a value of each parameter included in each piece of sensing data is one. For example, taking each piece of sensing data including the values of the six parameters as an example, each piece of sensing data includes a temperature value, a humidity value, a brightness value, a nitrogen value, a phosphorus value, and a potassium value.

In one embodiment, the acquisition module 301 acquires the data sensed by the M number of sensing devices 33 once in each of the T growth periods.

In one embodiment, each set of growth data includes the values of the N number of parameters of the plant 4 in the T growth periods. The T number of sensing data included in each set of growth data refers to the values of various parameters sensed by each sensing device 33 during the T growth periods. Each piece of sensing data corresponds to the values of various parameters of the plant 4 in one of the growth periods.

At block S2, the execution module 302 determines one group of growth data as reference data from the M groups of growth data. The execution module 302 regards each group of growth data in the other M−1 groups of growth data except for the reference data as a group of data to be tested (hereinafter “detection data”), and thus the execution module 302 obtains the M−1 group of detection data.

In one embodiment, the execution module 302 may determine one group of growth data from the M groups of growth data as the reference data in response to user input. For example, the reference data may be set as the growth data corresponding to the best harvested plant 4.

At block S3, the execution module 302 determines a first effective range of each of the N number of parameters based on the T number of sensing data included in the reference data, and filters the reference data according to the first effective range of each parameter to obtain filtered reference data.

In one embodiment, a method of determining the first effective range of each of the N number of parameters based on the T number of sensing data included in the reference data includes:

Determining a central tendency quantity E0 of each parameter based on the T number of sensing data included in the reference data, and determining the first effective range of each parameter based on the central tendency quantity E0 of each parameter as [E1, E2], in which E1=E0−E0*X1; E2=E0+E0*X1, X1 is a preset coefficient.

In one embodiment, the central tendency quantity E0 of each parameter refers to the median of all the values corresponding to each parameter in the T number of sensing data included in the reference data.

In one embodiment, the reason why the median is used as the central tendency quantity E0 is that the average is susceptible to extreme values, and there may not be a mode.

In one embodiment, the execution module 302 may arrange all the values corresponding to any one of the parameters in the T number of sensing data included in the reference data in sequence from small to large. In response that the total number of all values corresponding to any one parameter is an odd number, the middle value is used as the central tendency quantity E0 of the parameter. In response that the total number of all values corresponding to any one parameter is an even number, the average of the two middle values is taken as the central tendency quantity E0 of the parameter.

For example, assuming that the reference data includes a total of five pieces of sensing data and the parameter “nitrogen content” is arranged in order from small to large as 1.3, 1.3, 1.5, 1.8, 3.0, then the central tendency quantity E0 of “nitrogen content” is equal to 1.5.

For another example, assuming that the reference data includes a total of eight pieces of sensing data and the parameter “nitrogen content” is arranged in order from small to large as 0.8, 0.9, 1.3, 1.3, 1.5, 1.5, 1.8 and 3.0, then the central tendency quantity E0 of “nitrogen content” is equal to E0=((1.3+1.5)/2), that is, E0 is equal to 1.4.

In one embodiment, a method of filtering the reference data based on the first effective range of each parameter includes:

Obtaining all the values corresponding to each parameter in the reference data, and deleting the values that do not belong to the first effective range of the parameters.

For example, the first effective range of nitrogen is [0.75, 2.25], and the values corresponding to “nitrogen content” in the reference data are 0.5, 1.3, 1.5, 1.4, 1.8, 2.3. 0.5 is the nitrogen content in the first growth period, 1.3 is the nitrogen content in the second growth period, 1.5 is the nitrogen content in the third growth period, 1.4 is the nitrogen content in the fourth growth period, 1.8 is the nitrogen content in the fifth growth period, and 2.3 is the nitrogen content in the sixth growth period. Then, the execution module 302 deletes 0.5 and 2.3 because 0.5 and 2.3 are not within the first effective range [0.75, 2.25].

At block S4, the execution module 302 determines a second effective range of each parameter in each growth period based on the filtered reference data, and filters each of the M−1 groups of detection data based on the second effective range to obtain filtered detection data.

In one embodiment, the second effective range of each parameter in each growth period is [E1′, E2′], wherein E1′=V0−V0*X2; E2′=V0+V0*X2, and X2 is a preset coefficient. V0 represents the value of each parameter in each growth period in the filtered reference data.

For example, the nitrogen content values in the filtered reference data are 1.3, 1.5, 1.4, 1.8. 1.3 is the nitrogen content in the second growth period, 1.5 is the nitrogen content in the third growth period, 1.4 is the nitrogen content in the fourth growth period, and 1.8 is the nitrogen content in the fifth growth period. If the value of X2 is 0.1, then the second effective range of nitrogen content in the second growth period is 1.17-1.43, the second effective range of nitrogen content in the third growth period is 1.35-1.65, the second effective range of nitrogen content in the fourth growth period is 1.26-1.56, and the second effective range of nitrogen content in the fifth growth period is 1.62-1.98.

It should be noted that in block S3, in response that the value of a parameter in a growth period in the reference data is deleted, the second effective range of the deleted value is null.

In one embodiment, a method of separately filtering each of the M−1 groups of detection data based on the second effective range of each parameter in each growth period includes:

Deleting any sensing data in the T number of sensing data in each set of detection data in any growth period in response that none of the values of the N number of parameters in the sensing data are within the second effective range.

For example, sensing data D1 included in a group of detection data G1 is: 36.0 (temperature), 64 (humidity), and the sensing data D1 corresponds to the first growth period of the plant 4. That is, the temperature of the environment where the plant 4 is located during the first growth period is 36 degrees, and the humidity is 64 g/m3. To illustrate the present disclosure clearly and simply, only two parameters are taken as examples. If the second effective range of the parameter “temperature” in the first growth period is 38 to 41, and the second effective range of the parameter “humidity” in the first growth period is 65 to 66, then the execution module 302 deletes the sensing data D1 from the group of detection data G1 because the temperature of the sensing data D1 and the humidity of the sensing data D1 are not within the corresponding second effective range.

In one embodiment, if the second effective range of a parameter in a growth period is a null value, the execution module 302 determines that the value of the parameter corresponding to the growth period in each set of detection data falls within the second effective range of the parameter in the growth period. The parameter is any one of the N number of parameters, and the growth period is any one of the T number of growth periods.

For example, if the second effective range of the nitrogen content in the second growth period is a null value, then the execution module 302 may directly determine that the nitrogen content in the second growth period included in each set of detection data falls within the second effective range of the second growth period.

At block S5, the execution module 302 analyzes the filtered M−1 groups of detection data and obtains standard values of the N number of parameters in the T growth periods.

In one embodiment, a method of analyzing the filtered M−1 groups of detection data and obtaining the standard values of the N number of parameters in the T growth periods includes steps (a1)-(a3):

(a1) Obtaining all the sensing data included in the filtered M−1 groups of detection data and classifying all the obtained sensing data according to the growth period, thereby obtaining the sensing data corresponding to each growth period.

(a2) Determining the number of effective values for each classified sensing data corresponding to each growth period.

In one embodiment, the effective value refers to the value of the parameter falling in the second effective range.

For example, the growth period corresponding to a sensing data D2 is the second growth period, and the sensing data D2 includes the values of six parameters, which are 36.0 (temperature), 65 (humidity), 30 (brightness), 2.2 (nitrogen), 1.5 (phosphorus), 0.3 (potassium). If the values of the six parameters fall within the corresponding second effective range, the number of effective values in the sensing data D2 is 6. If the values of only five of the six parameters fall within the corresponding second effective range, the number of effective values in the sensing data D2 is 5.

(a3) Determining the sensing data of any growth period including the most number of effective values as target data, obtaining the values of the N number of parameters of the target data, and setting the obtained values of the N number of parameters of the target data as the standard values of the N number of parameters in the growth period.

For example, there are five pieces of sensing data corresponding to the second growth period, and each piece of sensing data includes values of the six parameters (temperature, humidity, brightness, nitrogen, phosphorus, and potassium). Among the five pieces of sensing data, a sensing data D3 includes six effective values, which are 36.0 (temperature), 65 (humidity), 30 (brightness), 2.2 (nitrogen), 1.5 (phosphorus), 0.3 (potassium), that is, the values of the six parameters included in the sensing data D3 all fall into the corresponding second effective ranges. Then, the execution module 302 determines the values of the six parameters included in the sensing data D3 as the standard values of the six parameters in the second growth period. Therefore, the execution module 302 sets the standard value of the parameter “temperature” in the second growth period to 36 degrees, sets the standard value of the parameter “humidity” in the second growth period to 65, sets the standard value of the parameter “brightness” in the second growth period to 30, sets the standard value of the parameter “nitrogen” in the second growth period to 2.2, sets the standard value of the parameter “phosphorus” in the second growth period to 1.5, and sets the standard value of the parameter “potassium” in the second growth period to 0.3.

It should be noted that if there is more than one piece of sensing data having the most effective values at the same time, the execution module 302 can randomly select from the multiple pieces of sensing data and set the selected piece of sensing data as the target data.

For example, of the five pieces of sensing data, two pieces of sensing data D1 and D3 each include five effective values, and the other three pieces of sensing data each include less than five effective values, then the execution module 302 can randomly select D1 or D3 as the target data.

It should be noted that blocks S1-S5 describe how to determine the standard value of each parameter in each growth period based on the multiple sets of growth data obtained from historically planted plants 4. The following block S6 introduces how to regulate the demand of each parameter of the plants 4 based on the above-determined standard values of the parameters in each growth period during a next planting cycle of the plants 4.

At block S6, the execution module 302 obtains the value of any one of the parameters of the plant 4 in any one of the T growth periods and compares the obtained value with the standard value of the corresponding parameter in the corresponding growth period. If the obtained value of the parameter is inconsistent with the standard value of the parameter in the corresponding growth period, the corresponding supply device 41 is adjusted. The supply devices 41 correspond to the aforementioned parameters.

For example, when the sensing device 33 in a certain growth period senses that the temperature of the plant 4 is lower than the standard value of the temperature corresponding to the certain growth period, the execution module 302 turns on the supply device 41, such as a heating device, until the temperature sensed by the sensing device 33 reaches the standard value, and then the heating device is turned off. The execution module 302 can also send out a warning message to alert an operator to check the supply device 41 on site.

In the several embodiments provided by the present disclosure, it should be understood that the disclosed device and method may be implemented in other ways. For example, the device embodiments described above are merely illustrative. For example, the division of the modules is only a logical function division, and there may be other division methods in actual implementation.

The modules described as separate components may or may not be physically separated, and the components displayed as modules may or may not be physical units, that is, they may be located in one place, or they may be distributed on multiple network units. Some or all of the modules can be selected according to actual needs to achieve the objectives of the solutions of the embodiments.

In addition, the functional modules in the various embodiments of the present disclosure may be integrated into one processing unit, or each unit may exist alone physically, or two or more units may be integrated into one unit. The above-mentioned integrated unit can be implemented in the form of hardware, or in the form of hardware plus software functional modules.

For those skilled in the art, it is obvious that the present disclosure is not limited to the details of the foregoing exemplary embodiments, and the present disclosure can be implemented in other specific forms without departing from the spirit or basic characteristics of the present disclosure. Therefore, no matter from which point of view, the embodiments should be regarded as exemplary and non-limiting. The scope of the present disclosure is defined by the appended claims rather than the above description, and therefore it is intended to fall into the claims. All changes within the meaning and scope of the equivalent parameters are included in the present disclosure. Any reference signs in the claims should not be regarded as limiting the claims involved. In addition, it is obvious that the word “including” does not exclude other units or steps, and the singular does not exclude the plural. The units or devices stated in the device claims can be implemented by software or hardware. Words such as first and second are used to denote names, but do not denote any specific order.

The embodiments shown and described above are only examples. Even though numerous characteristics and advantages of the present technology have been set forth in the foregoing description, together with details of the structure and function of the present disclosure, the disclosure is illustrative only, and changes may be made in the detail, including in matters of shape, size and arrangement of the parts within the principles of the present disclosure up to, and including, the full extent established by the broad general meaning of the terms used in the claims.

What is claimed is:

1. A plant growth monitoring method comprising:
sensing growth data of a plant by an M number of sensing devices, and obtaining M groups of growth data, each group of growth data comprising a T number of sensing data, each piece of sensing data in the T number of sensing data being associated with a growth period of a T number of growth periods of the plant, and each piece of sensing data in the T number of sensing data comprising values of an N number of parameters;
determining one group of the M number of groups of growth data as reference data, setting each group of growth data in the other M−1 groups of growth data except for the reference data as a group of detection data to be tested, and obtaining an M−1 number of groups of detection data
determining a first effective range of each of the N number of parameters based on the T number of sensing data of the reference data, filtering the reference data according to the first effective range of each parameter, and obtain filtered reference data;
determining a second effective range of each parameter in each growth period based on the filtered reference data, separately filtering each of the M−1 groups of detection data based on the second effective range of each parameter in each growth period, and obtain filtered detection data; and
analyzing the filtered M−1 groups of detection data and obtaining standard values of the N number of parameters in the T growth periods.

2. The plant growth monitoring method of claim 1, further comprising:
obtaining the value of any one of the parameters of the plant in any one of the T growth periods and comparing the obtained value with the standard value of the corresponding parameter in the corresponding growth period; and
in response that the obtained value of the parameter is inconsistent with the standard value of the parameter in the corresponding growth period, adjusting a corresponding supply device, the supply device corresponding to the parameter.

3. The plant growth monitoring method of claim 1, wherein determining the first effective range of each of the N number of parameters based on the T number of sensing data comprised in the reference data comprises:
determining a central tendency quantity E0 of each parameter based on the T number of sensing data of the reference data, and determining the first effective range of each parameter based on the central tendency quantity E0 of each parameter as [E1, E2]; wherein:
E1=E0−E0*X1;
E2=E0+E0*X1; and
X1 is a preset coefficient.

4. The plant growth monitoring method of claim 3, wherein filtering the reference data according to the first effective range of each parameter comprises:
obtaining all values corresponding to each parameter in the reference data, and deleting the values that do not belong to the first effective range of the parameters.

5. The plant growth monitoring method of claim 3, wherein:
the second effective range of each parameter in each growth period is [E1′, E2′]; wherein:
E1′=V0−V0*X2;
E2′=V0+V0*X2;
X2 is a preset coefficient; and
V0 represents the value of each parameter in each growth period in the filtered reference data.

6. The plant growth monitoring method of claim 5, wherein separately filtering each of the M−1 groups of detection data based on the second effective range of each parameter in each growth period comprises:
deleting any sensing data in the T number of sensing data in each set of detection data in any growth period in response that none of the values of the N number of parameters in the sensing data are within the second effective range.

7. The plant growth monitoring method of claim 6, wherein analyzing the filtered M−1 groups of detection data and obtaining the standard values of the N number of parameters in the T growth periods comprises:
obtaining all the sensing data of the filtered M−1 groups of detection data and classifying all the obtained sensing data according to the growth period, and obtaining the sensing data corresponding to each growth period;
determining the number of effective values for each piece of classified sensing data corresponding to each growth period; and
determining the sensing data of any growth period having the most number of effective values as target data, obtaining the values of the N number of parameters of the target data, and setting the obtained values of the N number of parameters of the target data as the standard values of the N number of parameters in the growth period.

8. A monitoring device comprising:
a processor; and
a memory storing a plurality of instructions, which when executed by the processor, cause the processor to:
sense growth data of a plant by an M number of sensing devices, and obtain M groups of growth data, each group of growth data comprising a T number of sensing data, each piece of sensing data in the T number of sensing data being associated with a growth period of a T number of growth periods of the plant, and each piece of sensing data in the T number of sensing data comprising values of an N number of parameters;
determine one group of the M number of groups of growth data as reference data, set each group of growth data in the other M−1 groups of growth data except for the reference data as a group of detection data to be tested, and obtain an M−1 number of groups of detection data;
determine a first effective range of each of the N number of parameters based on the T number of sensing data of the reference data, filter the reference data according to the first effective range of each parameter, and obtain filtered reference data;
determine a second effective range of each parameter in each growth period based on the filtered reference data, separately filter each of the M−1 groups of detection data based on the second effective range of each parameter in each growth period, and obtain filtered detection data; and
analyze the filtered M−1 groups of detection data and obtain standard values of the N number of parameters in the T growth periods.

9. The monitoring device of claim 8, wherein the processor is further configured to:
obtain the value of any one of the parameters of the plant in any one of the T growth periods and compare the obtained value with the standard value of the corresponding parameter in the corresponding growth period; and
in response that the obtained value of the parameter is inconsistent with the standard value of the parameter in the corresponding growth period, adjust a corresponding supply device, the supply device corresponding to the parameter.

10. The monitoring device of claim 8, wherein a method of the processor determining the first effective range of each of the N number of parameters based on the T number of sensing data comprised in the reference data comprises:
determining a central tendency quantity E0 of each parameter based on the T number of sensing data of the reference data, and determining the first effective range of each parameter based on the central tendency quantity E0 of each parameter as [E1, E2]; wherein:
E1=E0−E0*X1;
E2=E0+E0*X1; and
X1 is a preset coefficient.

11. The monitoring device of claim 10, wherein a method of the processor filtering the reference data according to the first effective range of each parameter comprises:
obtaining all values corresponding to each parameter in the reference data, and deleting the values that do not belong to the first effective range of the parameters.

12. The monitoring device of claim 10, wherein:
the second effective range of each parameter in each growth period is [E1′, E2′]; wherein:
E1′=V0−V0*X2;
E2′=V0+V0*X2;
X2 is a preset coefficient; and
V0 represents the value of each parameter in each growth period in the filtered reference data.

13. The monitoring device of claim 12, wherein a method of the processor separately filtering each of the M−1 groups of detection data based on the second effective range of each parameter in each growth period comprises:
deleting any sensing data in the T number of sensing data in each set of detection data in any growth period in response that none of the values of the N number of parameters in the sensing data are within the second effective range.

14. The monitoring device of claim 13, wherein a method of the processor analyzing the filtered M−1 groups of detection data and obtaining the standard values of the N number of parameters in the T growth periods comprises:
obtaining all the sensing data of the filtered M−1 groups of detection data and classifying all the obtained sensing data according to the growth period, and obtaining the sensing data corresponding to each growth period;
determining the number of effective values for each piece of classified sensing data corresponding to each growth period; and
determining the sensing data of any growth period having the most number of effective values as target data, obtaining the values of the N number of parameters of the target data, and setting the obtained values of the N number of parameters of the target data as the standard values of the N number of parameters in the growth period.

15. A plant factory comprising:
a monitoring device;
an M number of sensing devices each communicating with the monitoring device, each sensing device configured to sense values of an N number of parameters of a plant in each growth period of the plant; and
at least one supply device for adjusting the N number of parameters to the plant.
